(19) United States
(12) Patent Application Publication
(10) Pub. No.: US 2019/0231841 A1
(43) Pub. Date: Aug. 1, 2019

(54) CENCHRUS CILIARIS L. AS AN ANTICANCER AGENT

(71) Applicant: KING SAUD UNIVERSITY, RIYADH (SA)

(72) Inventors: AMANI SHAFEEK AWAAD, RIYADH (SA); NOURAH AHMED AL QURAIN, RIYADH (SA); HAYA FAHD ALKANHAL, RIYADH (SA); REHAM MOSTAFA EL-MELIGY, RIYADH (SA); FATMAH ALI AL-ASAMARY, RIYADH (SA)

(21) Appl. No.: 15/882,929

(22) Filed: Jan. 29, 2018

(51) Int. Cl. A61K 36/899; A61P 35/00

(52) U.S. Cl. CPC A61K 36/899; A61P 35/00

(57) ABSTRACT

Cenchrus ciliaris L. as an anticancer agent was obtained by separately extracting the aerial parts of the plant and the root part of the plants. Extractions were performed first in ethanol, and then a portion successively in water, chloroform, and aqueous butanol. All extracts were tested for cytotoxicity against six cancer cell lines. Generally, the root extracts exhibited greater cytotoxicity than the aerial extracts. The highest anticancer effects for both aerial and root extracts were shown against hepatocellular cancer, colorectal cancer, and lung cancer, compared to the other cell lines tested. The chloroform extracts showed greater anticancer effects, both for aerial and root extracts.

### BACKGROUND

### 1. Field

The disclosure of the present patent application relates to natural products having anticancer effects, and particularly to Cenchrus ciliaris L. as an anticancer agent.

### 2. Description of the Related Art

Cancer is a multifactorial disease with a multistep pathogenesis. Based on National Cancer Institute (NIH) publications and announcements, available treatments for cancer depend on the type of cancer and include surgery, radiation therapy, chemotherapy, immunotherapy, targeted therapy, hormone therapy, stem cell therapy, and precision medicine. However, these treatments have multiple side effects that can affect mortality rates. A Global Snapshot done by the World Health Organization (WHO) in 2015 states that cancer is a leading cause of mortality and morbidity globally, affecting more than 14 million people annually. Cancer cells are known to continuously develop resistance to current treatments, and for that reason, the need for new treatments arises.

Natural products have been used extensively as a source of anticancer compounds. Multiple anticancer “lead” compounds have been isolated and identified from many different natural sources. After chemical modifications and bioevaluations, these compounds have given rise to important drugs. Many pharmaceutical agents have been discovered by the testing of natural products found in microorganisms, animals, plants, and marine organisms. Irinotecan, vincristine, paclitaxel, and etoposide are plant-derived compounds that have been used for the treatment of cancer. Plants produce a large variety of secondary metabolites that can exceed a hundred thousand molecules, and the promising anticancer effects of plants intensifies the need for investigational studies on plants.

Cenchrus ciliaris L. (Buffel grass) is a pasture grass belonging to the Poaceae family, formerly called “Gramineae”. Poaceae is very big family that includes many genera and species with a variety of chemical content and biological activity, such as anticancer, increasing milk production, antifungal, antibacterial, anthelmintic, anti-amoebic, Cyclooxygenase (COX) I and II inhibitory activity, dysmenorrheal, and uterine-relaxing activity. In view of the severe side effects of surgery, chemotherapy, radiation therapy, and other treatment regimens for cancer, it may be useful to consider the anticancer potential of natural products having less severe side effects.

Thus, Cenchrus ciliaris L. as an anticancer agent solving the aforementioned problems is desired.

### SUMMARY

Cenchrus ciliaris L. as an anticancer agent was obtained by separately extracting the aerial parts of the plant and the root part of the plants. Extractions were performed first in ethanol, and then a portion successively in water, chloroform, and aqueous butanol. All extracts were tested for cytotoxicity against six cancer cell lines. Generally, the root extracts exhibited greater cytotoxicity than the aerial extracts. The highest anticancer effects for both aerial and root extracts were shown against hepatocellular cancer, colorectal cancer, and lung cancer, compared to the other cell lines tested. The chloroform extracts showed greater anticancer effects, both for aerial and root extracts.

These and other features of the present disclosure will become readily apparent upon further review of the following specification.

### DETAILED DESCRIPTION OF THE PREFERRED EMBODIMENTS

Cenchrus ciliaris L. as an anticancer agent was obtained by separately extracting the aerial parts of the plant and the root part of the plants. Extractions were performed first in ethanol, and then a portion successively in water and chloroform, followed by extraction of the remaining aqueous extract (after removal of the chloroform extracts) in n-butanol (saturated with water). All extracts were tested for cytotoxicity against six cancer cell lines. Generally, the root extracts exhibited greater cytotoxicity than the aerial extracts. The highest anticancer effects for both aerial and root extracts were shown against hepatocellular cancer, colorectal cancer, and lung cancer, compared to the other cell lines tested. The chloroform extracts showed greater anticancer effects, both for aerial and root extracts.

In the following examples, Cenchrus ciliaris L. aerial and root parts were collected in March 2017, from the region of Alkharj in Saudi Arabia and identified by Dr. Jacob Thomas, Taxonomist, College of Science, King Saud University. The plant materials of aerial parts and root were air-dried, ground to powder, and packed and stored in a tightly closed container for phytochemical analysis and testing biological activity.

### Example 1

### Extraction—General Procedure

The air-dried powdered plant material, 1 kg each of aerial and root parts, were separately extracted by percolation in 2 liter of ethanol (95%) for 2 days and the solvents of each part (aerial and root) were filtered over filter paper, and the marc lifted of each part (aerial and root) was extracted four times by the same method. The total alcohol extracts were concentrated at a temperature not exceeding 35° C. The obtained alcohol-free extracts were symbolized as; A & R for Arial parts and root respectively).

The total alcohol-free extracts (A & R) were separately dissolved in hot water and filtered using a piece of cotton. The non-filtered parts were symbolized as AL, an indication for lipoidal matters of the aerial parts only. No lipoidal matter was obtained from the root parts. The aqueous filtered portions were symbolized by AP and RP (for aerial and root portions, respectively).

The aqueous fraction residues (AP and RP) were separately extracted successively using chloroform and butanol saturated with water. Each extract was passed over anhydrous sodium sulfate and evaporated using a rotatory evaporator and low temperature to obtain residual gummi extracts with different weights. The obtained dry chloroform extracts were symbolized as APC and RPC for aerial parts & root parts, respectively, while the butanol extracts were symbolized as APB and RPB for aerial parts & root parts, respectively. During butanol extraction, one resinous compound each was isolated from APB and from RPB. These compounds were symbolized as AR and RR for aerial and root, respectively.

### Example 2

### Extraction—Detailed Procedure for Aerial Parts

In the first step, Cenchrus Ciliaris L. aerial parts were freshly collected and washed (to get rid of any sand or dust attached) and dried using a hot air steam drying machine, as follows. The plant materials were spread out on shallow trays, which are placed on mobile racks and passed into a tunnel where they meet a stream of warm air using constant temperature at 20-40° C., then ground to powder. In the second step, the plant material (1000 g of Cenchrus Ciliaris L. aerial parts) was extracted by percolation in 2 liter of aqueous ethanol (90%) for 2 days and the solvent was filtered over filter paper, and then the marc was lifted and extracted again four times by the same method. In the third step, the collected total alcohol extracts obtained from step 2 were concentrated using a rotatory evaporator at temperatures not exceeding 25° C. About 50.3 g of oily residue was obtained from this step.

In the fourth step, the obtained oily residue (50.3 g) was then dissolved in de-ionized distilled water (300 ml) and filtered using MicroFunnel™ Filter Funnels with the aid of a suction pump to speed the filtration and to prevent microbial contamination (the process was carried out under laminar flow for optimum sterile conditions). Wherefrom, we got two extracts, a non-filtered (solid) part_(lipoidal compounds) and a filtered part_(aqueous extract). In the fifth step, the non-filtrate parts from step 4 contain the lipoidal compounds (fatty acids, chlorophyll, sterols and triterpenes), which were re-purified by dissolving in distilled water and were re-filtered as before (in step 4) to remove any remaining soluble compounds that may be attached to it. The residue obtained (5.24 g) was then kept for evaluation of its effect on cancer cells. In the sixth step, the obtained filtered part_(aqueous extract) from step 4 was extracted till exhaustion using chloroform (analytical grade). The collected chloroform extracts were filtered over anhydrous sodium sulfate to get rid of any trace of water. The extract was then dried of chloroform using reduced pressure at temperatures not exceeding 20° C. to obtain low polarity phenolic compound matter (some flavonoids and coumarin) with a weight of 10.9 g.

In the seventh step, after removal of low polarity phenolic compounds matter in step 6, the remaining aqueous extract was extracted using n-butanol (saturated with water) till exhaustion. The extracts were filtered over anhydrous sodium sulfate to get rid of any trace of water. The extract was then dried of n-butanol using reduced pressure at temperatures not exceeding 35° C. to obtain the highly polar phenolic and non-phenolic compounds (carbohydrates and/or glycosides, flavonoids, proteins and/or amino acids, phenolic compounds and tannins) with a weight of 14.5 g. In the eighth step, residues obtained from step 7 (the highly polar phenolic and non-phenolic compounds) were dissolved in absolute ethanol and filtered using filter paper. The non-filtered (solid) parts were collected in a pure container and left to dry in a dissector with an air vacuum pump. After three days, the obtained resinous compound was weighed to produce 8.9 g. The filtered extract was dried using reduced pressure at temperatures not exceeding 35° C. to obtain the oily residue, which was symbolized as n-butanol extract.

### Example 3

### Extraction—Detailed Procedure for Root Parts

In the first step, Cenchrus Ciliaris L. root parts were freshly collected and washed to get rid of any sand or dust attached. The plant materials were spread out on shallow trays, which are placed on mobile racks and passed into a tunnel where they meet a stream of warm air using constant temperature at 50-55° C., then ground to powder. In the second step, the plant material (1000 g of Cenchrus Ciliaris L. root parts) was extracted by percolation in 2 liters of aqueous ethanol (90%) for 2 days. The solvent was filtered over filter paper. The marc was lifted and extracted again for four times by the same method. In the third step, the collected total alcohol extracts obtained from step 2 were concentrated using a rotatory evaporator at temperatures not exceeding 25° C. About 40.5 g of oily residue was obtained from this step.

In the fourth step, the obtained oily residue (40.5 g) was then dissolved in distilled water (250 ml) and filtered using MicroFunnel™ Filter Funnels with the aid of a suction pump to speed the filtration and to prevent microbial contamination (the process was carried out under laminar flow for optimum sterile conditions). Wherefrom, we got one extract; the filtered part_(aqueous extract). No lipoidal compound was obtained. In the fifth step, the obtained filtered part_(aqueous extract) from step 4 was extracted till exhaustion using chloroform (analytical grade). The collected chloroform extracts were filtered over anhydrous sodium sulfate to get rid of any trace of water. The extract was then dried of chloroform using reduced pressure at temperatures not exceeding 20° C. to obtain low polarity phenolic compounds (some flavonoids and coumarin) with a weight of 7.8 g. In the sixth step, after removal of the low polarity phenolic compounds in step 5, the aqueous extract was extracted using n-butanol (saturated with water) till exhaustion. The extracts were filtered over anhydrous sodium sulfate to get rid of any trace of water. The extract was then dried of n-butanol using reduced pressure at temperatures not exceeding 35° C. to obtain the highly polar phenolic and non-phenolic compounds (carbohydrates and/or glycosides, flavonoids, proteins and/or amino acids, phenolic compounds and tannins) with a weight of 15.3 g.

In the seventh step, residues obtained from step 6 (the highly polar phenolic and non-phenolic compounds) were dissolved in absolute ethanol and filtered using filter paper. The non-filtered (solid) parts were collected in a pure container and left to dry in a dissector with an air vacuum pump. After three days, the obtained resinous compound was weighed to produce 6.6 g. The filtered extract was dried using reduced pressure at temperatures not exceeding 35° C. to obtain the oily residue, which symbolized as n-butanol extract.

### Example 4

### Biological Testing—General Materials and Procedures

The cell lines A-549 (Lung carcinoma), CACO (Colorectal carcinoma), HCT-116 (Colon carcinoma), Hela (Cervical carcinoma), HepG-2 (Hepatocellular carcinoma), and MCF-7 (Breast carcinoma) were obtained from the Regional Center for Mycology and Biotechnology, Al-Azhar University, Cairo, Egypt.

The antitumor assay was carried out on the above cell lines as published by A Ooi et al., ‘A Guide to Transient Expression of Membrane Proteins in HEK-293 Cells for Functional Characterization”, Front. Physiol. 7:300, published online 19 Jul. 2016, for all of the extracts which have been obtained in section above (A, R, AL, APC, RPC, APB, RPB, AR &RR). Briefly, the cell lines were suspended in medium at a concentration of 5×104 cell/well in Corning® 96-well tissue culture plates and then incubated for 24 hr. The tested extracts were then added into 96-well plates (six replicates) to achieve seven concentrations for each extract. Six vehicle controls with media or 0.5% DMSO were run for each 96-well plate as a control. After incubation for 24 h, the numbers of viable cells were determined by the MTT assay method. The media was then removed from the 96 well plate and replaced with 100 μl of fresh culture RPMI 1640 medium without phenol red, then 10 μl of the 12 mM MTT (Sigma) stock solution (5 mg of MTT in 1 mL of PBS) was added to each well, including the untreated controls. The 96-well plates were then incubated at 37° C. and 5% CO₂ for 4 hours. An 85 μl aliquot of the media was removed from the wells, and 50 μl of DMSO was added to each well and mixed thoroughly with the pipette and incubated at 37° C. for 10 min. Then, the optical density was measured at 590 nm with the microplate reader to determine the number of viable cells. The percentage of viability was calculated as: (ODt/ODc)×100%, where ODt is the mean optical density of wells treated with the tested sample and ODc is the mean optical density of untreated cells.

The relation between surviving cells and extract concentration is plotted to get the survival curve of each tumor cell line after treatment with the specified extract. The 50% inhibitory concentration (IC50) (the concentration required to cause toxic effects in 50% of intact cells) was estimated from graphic plots of the dose response curve for each concentration using Graphpad Prism software (San Diego, Calif. USA).

Swiss albino mice of both sexes (30-35 g) were purchased from Alazahar University animal house, Cairo, Egypt. The animals were kept in standard polypropylene cages and maintained under standard conditions.

Each dried alcohol extract of Cenchrus ciliaris (aerial parts and root parts separately) was suspended in distilled water. Freshly, just before administration, a few drops of Tween 80 were used as emulsifying agent.

For the Acute Toxicity (LD50) test, each dried alcohol extract of Cenchrus ciliaris (aerial parts and root parts separately) was orally given to the animal for median lethal dose (LD50), as described by El-Meligy et al., “Prophylactic and curative anti-ulcerogenic activity and the possible mechanisms of action of some desert plants”, Saudi Pharmaceutical Journal 25, 387-396 (2017).

For determination of sub-chronic toxicity, rats were divided into 2 groups each of 6 rats. The 1st group was administrated the vehicle orally and left as a control, while 23 groups were separately administrated the total alcohol extract of aerial parts or root parts, respectively, in a dose of 400 mg/kg for 15 days. After the examination period, the collected sera were used for determination of liver and kidney functions.

For statistical analysis, all values were expressed as mean±S.D. Comparisons between means were carried out using a one-way ANOVA test, followed by the Tukey HSD test using SPSS, version 14 (SPSS, Chicago, Ill.). Differences at p=50.05 were considered statistically significant.

### Example 5

### Phytochemical Screening

Phytochemical screening of Cenchrus ciliaris L. showed the presence of the following groups: carbohydrates and/or glycosides, flavonoids, sterols and/or triterpenes, anthraquinones, protein and/or amino acids, and tannins and absence of saponin, alkaloids, cardinolides, and oxidase enzyme in all the investigated parts.

### Example 6

### Total Yield

Total alcohol extracts were 30.3 & 40.5 for aerial part and root respectively (A, R). The lipoidal matter was only obtained from the Arial parts (AL) and it gave 3.24 g. Chloroform extracts evaporation produced 10.9 and 7.8 g (from aerial parts & root parts, respectively). But butanol evaporation gave 14.5 and 15.3 g from aerial parts & root parts, respectively. The isolated resinous matter collected was 8.9 and 16.6 g, respectively (AR and RR).

### Example 7

### Anticancer Activity

The in vitro antitumor activities of Cenchrus ciliaris L. extracts were evaluated on six cell lines. The obtained results exhibited direct cytotoxic effect (Table 1) with variable inhibiting effect on the growth of the listed cell lines, compared to vinblastine sulfate as a reference standard drug. The direct cytotoxic effects showed different IC₅₀ values, ranging from 11.1±0.3 to 267±14.6 μg/ml.

In general, all root extracts showed the best activity against most of the tested cell lines, especially HepG-2 (Hepatocellular carcinoma) (9±2.1 μgimp, which was somewhat closely related to the effect of vinblastine sulfate (2.93±0.3 μg/ml).

The highest anticancer effect of Cenchrus ciliaris L aerial parts and root part extracts were recorded on HepG-2 (Hepatocellular carcinoma), CACO (colorectal carcinoma), and A-549 (Lung carcinoma), which were better than the standard drug, especially in the case of the anticancer effect on CACO (colorectal carcinoma) and on A-549 (Lung carcinoma).

Chloroform extracts of both aerial and root parts achieved the best anticancer activities on all of the cell lines, especially with colorectal cancer (CACO) and Lung carcinoma (A-549). The effect of chloroform root extract was 11.1±0.3 μg/ml on colorectal (CACO) and 20.5±0.6 μg/ml on Lung carcinoma (A-549), respectively, while in the case of aerial chloroform extract the effect was 14.5±0.7& 27.2±1.6 μg/ml on colorectal cancer (CACO) and Lung carcinoma (A-549), respectively, and all of these effects were better than vinblastine sulfate (24.6±0.7 and 30.3±1.4 μg/ml for colorectal and Lung carcinoma, respectively). Phenolic compounds are known to possess antioxidant and anticancer activity.

TABLE 1
IC50 (μg/ml) for selected cancer cell lines

| | | Cell line | | | | | |
|---|---|---|---|---|---|---|---|
| | | A-549 | CACO | HCT-116 | Hela | HepG-2 | MCF-7 |
| | | (Lung | (Colorectal | (Colon | (Cervical | (Hepatocellular | (Breast |
| Standard/Extract | | carcinoma) | carcinoma) | carcinoma) | carcinoma) | carcinoma) | carcinoma) |
| Vinblastine sulfate | | 24.6 ± 0.7 | 30.3 ± 1.4 | 3.5 ± 0.2 | 59.7 ± 2.1 | 2.93 ± 0.3 | 5.9 ± 0.4 |
| Aerial | A | 124 ± 6.2 | 168 ± 9.4 | 248 ± 13.8 | 204 ± 8.6 | 40.5 ± 3.9 | 78.2 ± 7.6 |
| extract | AL | 59.7 ± 2.3 | 40.6 ± 2.6 | 54 ± 2.9 | 107 ± 6.3 | 30.9 ± 2.8 | 58.4 ± 4.3 |
| | APC | 14.5 ± 0.7 | 27.2 ± 1.6 | 24.5 ± 0.9 | 27.5 ± 1.4 | 12 ± 0.8 | 50.8 ± 3.4 |
| | APB | 48.9 ± 4.2 | 55.8 ± 3.4 | 267 ± 14.6 | 123 ± 8.3 | 51.2 ± 5.7 | 237 ± 9.8 |
| | AR | 45.2 ± 3.7 | 22.5 ± 3.1 | 154 ± 6.4 | 162 ± 8.1 | 23.6 ± 3.2 | 121 ± 4.7 |
| Root | R | 11.1 ± 0.3 | 20.5 ± 0.6 | 18.8 ± 2.9 | 22.6 ± 2.4 | 9 ± 2.1 | 46.5 ± 2.6 |
| extract | RPC | 49.2 ± 1.7 | 28.8 ± 1.6 | 155 ± 6.3 | 113 ± 10.2 | 46.9 ± 3.4 | 51.5 ± 4.3 |
| | RPB | 43.9 ± 2.4 | 29.2 ± 2.4 | 234 ± 7.9 | 234 ± 4.9 | 28.2 ± 1.9 | 111 ± 3.7 |
| | RR | 11.1 ± 0.3 | 20.5 ± 0.6 | 18.8 ± 2.9 | 22.6 ± 2.4 | 9 ± 2.1 | 46.5 ± 2.6 |

A: total alcohol aerial parts; R: total alcohol root; AL: lipoidal matter aerial parts; APC: chloroform extract aerial parts; RPC: chloroform extract roots; APB; butanol extract aerial parts; RPB: butanol extract root; AR: Resinous Compound aerial parts; RR; Resinous Compound root.

### Example 8

### Toxicity Testing

After oral admiration of Cenchrus ciliaris L. alcohol extracts (aerial parts and root parts) with different doses up to 5000 mg/kg, the results did not produce any sign of toxicity or animal death during 24 h of observation. Therefore, LD₅₀ of the tested extracts, up to 5000 mg/kg, is considered safe for human use.

The sub-chronic toxicity tests also supported the safety of the plant extracts (aerial parts and root parts). The oral dosing (400 mg/kg), which was given to rats for 14 days, did not affect the levels of ALT, AST, total bilirubin, total proteins, albumin, urea, and creatinine as compared to the control (see Table 2). It means that the investigated extracts are not hepatotoxic.

TABLE 2
Results of liver and kidney function testing

| | | | | Parameter | | | |
|---|---|---|---|---|---|---|---|
| | | | Total bilirubin | Total protein | Albumin | Urea | Creatinine |
| Analyte | ALT (U/L) | AST (U/L) | (mg/dL) | (g/dL) | (g/dL) | (mg/dL) | (mg/dL) |
| Control | 61.23 ± 1.4 | 47.70 ± 1.3 | 1.60 ± 0.3 | 8.90 ± 0.5 | 3.7 ± 0.5 | 36.66 ± 0.2 | 0.48 ± 0.4 |
| Aerial parts | 60.41 ± 1.3 | 48.90 ± 1.6 | 1.6 ± 0.3 | 8.80 ± 0. 3 | 3.3 ± 0.2 | 35.33 ± 1.3 | 0.50 ± 0.3 |
| Root | 62.30 ± 1.2 | 46.12 ± 1.4 | 1.62 ± 0.4 | 8.75 ± 0.9 | 3.1 ± 0.6 | 36.13 ± 1.2 | 0.47 ± 0.5 |

Upon testing of aerial and root parts of Cenchrus ciliaris L., it can be concluded that extracts of the two parts were effective in inhibiting the growth of lung (A-549), intestinal (CACO), colon (HCT-116), cervical (Hela), hepatocellular (HepG-2), and breast (MCF-7) carcinomas with a high safety margin for human use.

It is to be understood that the Cenchrus ciliaris L. as an anticancer agent is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method for achieving an effect in a patient, comprising administering an effective amount of an extract of aerial parts of Cenchrus ciliaris L. in ethanol to the patient, wherein the effect is death of lung cancer cells or inhibiting the growth of a lung cancer tumor, death of colorectal (CACO) cancer cells or inhibiting the growth of a colorectal (CACO) cancer tumor, death of colon (HCT-116) cancer cells or inhibiting the growth of a colon (HCT-116) cancer tumor, death of cervical cancer cells or inhibiting the growth of a cervical cancer tumor, death of hepatocellular cancer cells or inhibiting the growth of a hepatocellular cancer tumor, or death of breast cancer cells or inhibiting the growth of a breast cancer tumor, wherein the extract of Cenchrus ciliaris L. comprises:
a mixture of lipoidal compounds separated from the ethanol extract by serial extraction in water to form an aqueous extract and filtration of the aqueous extract to remove the lipoidal compounds;
wherein the mixture of lipoidal compounds includes fatty acids, chlorophyll, sterols and triterpenes;
a mixture of low polarity phenolic compounds separated from the aqueous extract by serial extraction in chloroform after removal of the lipoidal compounds to form a chloroform extract containing the mixture of low polarity phenolic compounds;
a mixture of highly polar phenolic and remaining non-phenolic compounds separated from the aqueous extract by serial extraction in n-butanol saturated with water after removal of the lipoidal compounds and low polarity phenolic compounds to form a butanol extract containing the mixture of highly polar phenolic and remaining non-phenolic compounds; and
wherein the mixture of highly polar phenolic and remaining non-phenolic compounds includes compounds selected from the group consisting of carbohydrates, glycosides, flavonoids, proteins, amino acids, phenolic compounds and tannins.

2-6. (canceled)

7. The method for achieving an effect in a patient according to claim 1, wherein the mixture of low polarity phenolic compounds includes flavonoids and coumarin.

8-15. (canceled)

16. A method for achieving an effect in a patient, comprising administering an effective amount of an extract of root parts of Cenchrus ciliaris L. in ethanol to the patient, wherein the effect is death of lung cancer cells or inhibiting the growth of a lung cancer tumor, death of colorectal (CACO) cancer cells or inhibiting the growth of a colorectal (CACO) cancer tumor, death of colon (HCT-116) cancer cells or inhibiting the growth of a colon (HCT-116) cancer tumor, death of cervical cancer cells or inhibiting the growth of a cervical cancer tumor, death of hepatocellular cancer cells or inhibiting the growth of a hepatocellular cancer tumor, or death of breast cancer cells or inhibiting the growth of a breast cancer tumor, wherein the extract of Cenchrus ciliaris L. comprises:
a mixture of low polarity phenolic compounds separated from the aqueous extract by serial extraction in chloroform to form a chloroform extract containing the mixture of low polarity phenolic compounds;
a mixture of highly polar phenolic and remaining non-phenolic compounds separated from the aqueous extract by serial extraction in n-butanol saturated with water after removal of the low polarity phenolic compounds to form a butanol extract containing the mixture of highly polar phenolic and remaining non-phenolic compounds; and
wherein the mixture of highly polar phenolic and remaining non-phenolic compounds includes compounds selected from the group consisting of carbohydrates, glycosides, flavonoids, proteins, amino acids, phenolic compounds and tannins.
